(19) United States
(12) Patent Application Publication
(10) Pub. No.: US 2009/0325911 A1
(43) Pub. Date: Dec. 31, 2009

(54) Use of Androgens for the Treatment of Parkinson's Disease

(75) Inventors: Jin Xu, Wellesley, MA (US); Nan Zhong, Stony Brook, NY (US)

Correspondence Address: EDWARDS ANGELL PALMER & DODGE LLP, P.O. BOX 55874, BOSTON, MA, 02205, US

(73) Assignee: CARITAS ST. ELIZABETH MEDICAL CENTER OF BOSTON, INC.

(21) Appl. No.: 12/090,726

(22) PCT Filed: Oct. 20, 2006
(86) PCT No.: PCT/US06/41234

§ 371 Date: Mar. 10, 2009

Related U.S. Application Data
(60) Provisional application No. 60/729,117, filed on Oct. 21, 2005.

(51) Int. Cl. A61K 31/56; C12N 5/06; C12Q 1/68

(52) U.S. Cl. 514/169; 435/325; 435/6

(57) ABSTRACT

The invention generally provides therapeutic and prophylactic methods relating to the use of androgens for the treatment of Parkinson's disease or other neurodegenerative diseases. In addition, the invention provides related methods of screening for compounds for the treatment of Parkinson's disease.

### CROSS-REFERENCE TO RELATED APPLICATION

This application claims the benefit of the following U.S. Provisional Application No. 60/729,117, the entire contents of which is incorporated herein by reference.

### BACKGROUND OF THE INVENTION

Parkinson's disease is a common neurodegenerative disorder, second in prevalence only to Alzheimer disease. Parkinson's disease is a heterogeneous disease, and the majority of the cases of Parkinson's disease appear to have sporadic origins. Genetic analyses have identified a number of genes that contribute to Parkinson's disease susceptibility, either in an autosomal dominant or an autosomal recessive pattern. Mutations in PARK1 (alpha-synuclein), PARK2 (parkin), and PARK7 (DJ-1) genes have been shown to cause Parkinson's disease. Regardless of the underlying genetic causation, the symptoms of Parkinson's disease generally include slowed movement (bradykinesia), resting tremor, muscular rigidity, and postural instability. These clinical symptoms result from the near-total destruction of the nigrostriatal dopamine system, which regulates movement. Symptoms of the disease are typically controlled with medications that increase levels of brain dopamine, but these medications have a number of severe side effects. No cure is presently available for Parkinson's disease, and the disorder inevitably progresses to total disability, often accompanied by the general deterioration of all brain functions, and death. Given the inadequacy of current therapies, new methods for treating Parkinson's disease and other neurodegenerative diseases are urgently required.

### SUMMARY OF THE INVENTION

The invention generally provides therapeutic and prophylactic compositions and methods featuring androgens for the treatment of Parkinson's disease or other neurodegenerative diseases.

In a first aspect, the invention features a method for reducing neuronal cell death associated with a neurodegenerative disease (e.g., Parkinson's disease, Huntington's Disease, Kennedy's Disease, and spinocerebellar ataxia), the method involving contacting a cell (e.g., mammalian, such as human) at risk of cell death with an effective amount of an androgen (e.g., testosterone or dihydrotestosterone) or androgen analog thereby reducing neuronal cell death. In one embodiment, the method increases (e.g., by 5%, 10%, 25%, 50%, or 75%) tyrosine hydroxylase expression in the cell.

In yet another aspect, the invention features a method for reducing oxidative stress (e.g., oxidative stress is associated with ageing) in a cell in need thereof, the method involving contacting the cell with an androgen or androgen analog, thereby reducing oxidative stress.

In yet another aspect, the invention features a method for increasing tyrosine hydroxylase expression in a neuronal cell in need thereof, the method involving contacting the cell with an effective amount of an agent (e.g., valproic acid, sodium butyrate, trichostatin A, SAHA) that increases DJ-1 expression or activity.

In another aspect, the invention generally provides a method for treating a subject (e.g., human) having a neurodegenerative disease. The method involves administering to the subject an effective amount of an androgen, androgen analog, or fragment thereof that increases expression of a gene required for neuronal survival or maintenance. In one embodiment, the neurodegenerative disease is selected from the group consisting of Parkinson's disease, Huntington's disease, Kennedy's Disease, and spinocerebellar ataxia.

In a related aspect, the invention provides a method for treating or preventing Parkinson's disease in a subject. The method involves administering to the subject an effective amount of a compound that increases expression of tyrosine hydroxylase.

In another related aspect, the invention provides a method for enhancing dopamine synthesis in a subject. The method involves administering to the subject an effective amount of a compound that enhances expression of tyrosine hydroxylase.

In another aspect, the invention provides a method for enhancing cell survival in a neuronal cell (e.g., dopaminergic neuron) at risk of cell death. The method involves contacting the cell with an effective amount of an androgen, androgen analog, or fragment thereof, where the contacting increases expression of a gene required for neuronal survival or maintenance. In one embodiment, the risk of cell death is associated with a neurodegenerative disease selected from the group consisting of Parkinson's disease, Huntington's disease, Kennedy's Disease, and spinocerebellar ataxia.

In another aspect, the invention provides a method for enhancing cell survival in a neuronal cell at risk of cell death associated with Parkinson's disease. The method involves administering to the subject an effective amount of a compound that increases expression of tyrosine hydroxylase.

In a related aspect, the invention provides a method for enhancing dopamine synthesis in a neuronal cell. The method involves administering to the cell an effective amount of a compound that enhances expression of tyrosine hydroxylase.

In yet another aspect, the invention provides a method for identifying a compound useful for the treatment of a neurodegenerative disease. The method involves contacting a neuronal cell with a compound and an androgen receptor agonist; and identifying an increase in the expression of a gene of interest in the cell relative to a control cell not contacted with the candidate compound, where a compound that increases the expression of a gene of interest is a compound useful for the treatment of a neurodegenerative disease. In one embodiment, the gene of interest is any one or more of genes functioning in or regulating a mitochondrial activity, stress response, neuronal cell death, protein-folding, and neurotransmitter synthesis. Exemplary genes include tyrosine hydroxylase, which is involved in neurotransmitter synthesis, heat shock protein 70 (HSP 70), which is involved in protein folding and stress response, and glutamate cysteine ligase, which functions in cell death. In one embodiment, the increase in expression is detected at the level of transcription or translation.

In another aspect, the invention provides a method for identifying a compound useful for the treatment of Parkinson's disease. The method involves contacting a dopaminergic cell with a candidate compound and an androgen receptor agonist; and identifying an increase in tyrosine hydroxylase expression in the cell relative to a control cell not contacted with the candidate compound, where a compound that increases tyrosine hydroxylase expression is a compound useful for the treatment of a neurodegenerative disease.

In a related aspect, the invention provides a method for identifying a compound that increases tyrosine hydroxylase expression. The method involves contacting a dopaminergic cell with a compound and an androgen receptor agonist; and identifying an increase in tyrosine hydroxylase expression in the cell relative to a control cell not contacted with the candidate compound.

In another related aspect, the invention provides a method for identifying a compound that enhances cell survival in a dopaminergic cell at risk of cell death. The method involves: contacting a dopaminergic cell with a candidate compound and an androgen receptor agonist; and identifying an increase in tyrosine hydroxylase expression in the cell relative to a reference, where a compound that increases tyrosine hydroxylase expression is a compound that enhances cell survival.

In another aspect, the invention provides a method for identifying a gene required for neuronal survival or maintenance that is transcriptionally activated by androgen receptor binding. The method involves contacting a cell expressing the gene with an androgen receptor agonist; and identifying binding of the androgen receptor to a regulatory sequence present in the gene.

In yet another aspect, the invention provides a method for identifying a gene required for neuronal survival or maintenance that is transcriptionally activated by androgen receptor binding. The method involves contacting a cell expressing the gene with an androgen receptor agonist; and identifying an increase in expression of the gene in the cell relative to a control cell not contacted with the androgen receptor agonist.

In yet another aspect, the invention features a method for identifying a compound useful for the treatment of a neurodegenerative disease (e.g., Parkinson's disease, Huntington's Disease, Kennedy's Disease, and spinocerebellar ataxia), the method involving contacting a neuronal cell with a compound and an androgen receptor agonist; and identifying an increase in the expression of a gene of interest (e.g., genes regulating mitochondrial activities, stress responses, neuronal cell death, protein-folding, and neurotransmitter synthesis) in the cell relative to a control cell not contacted with the candidate compound, wherein a compound that increases the expression of a gene of interest is a compound useful for the treatment of a neurodegenerative disease. In one embodiment, the increase in expression is detected at the level of transcription or translation.

In yet another aspect, the invention features a method for identifying a compound useful for the treatment of Parkinson's disease, the method involving contacting a dopaminergic cell with a candidate compound and an androgen receptor agonist; and identifying an increase in tyrosine hydroxylase expression in the cell relative to a control cell not contacted with the candidate compound, wherein a compound that increases tyrosine hydroxylase expression is a compound useful for the treatment of a neurodegenerative disease.

In yet another aspect, the invention features a method for identifying a compound that increases tyrosine hydroxylase expression, the method involving contacting a dopaminergic cell with a compound and an androgen receptor agonist; and identifying an increase in tyrosine hydroxylase expression in the cell relative to a control cell not contacted with the candidate compound.

In yet another aspect, the invention features a method for identifying a compound that enhances cell survival in a dopaminergic cell at risk of cell death, the method involving contacting a dopaminergic cell with a candidate compound and an androgen receptor agonist; and identifying an increase in tyrosine hydroxylase expression in the cell relative to a reference, wherein a compound that increases tyrosine hydroxylase expression is a compound that enhances cell survival.

In yet another aspect, the invention features a method for identifying a gene required for neuronal survival or maintenance that is transcriptionally activated by androgen receptor binding, the method involving contacting a cell expressing the gene with an androgen receptor agonist; and identifying binding of the androgen receptor to a regulatory sequence present in the gene.

In yet another aspect, the invention features a method for identifying a gene required for neuronal survival or maintenance that is transcriptionally activated by androgen receptor binding, the method involving contacting a cell expressing the gene with an androgen receptor agonist; and identifying an increase in expression of the gene in the cell relative to a control cell not contacted with the androgen receptor agonist.

In yet another aspect, the invention features a kit for treating a neurodegenerative disease comprising an effective amount of an androgen or androgen analog. In one embodiment, the effective amount is sufficient to increase tyrosine hydroxylase expression or reduce cell death in a subject having a neurodegenerative disease.

In yet another aspect, the invention features a packaged pharmaceutical comprising an androgen or androgen analog; and instructions for using said androgen to treat a neurodegenerative disease. In various embodiments, the composition also contains a thereapeutic selected from the group consisting of deprenyl, amantadine, levodopa, carbidopa, entacapone, pramipexole, rasagiline, antihistamines, antidepressants, dopamine agonists, monoamine oxidase inhibitors (MAOIs), haloperidol, phenothiazine, reserpine, tetrabenazine, and co-enzyme Q10.

In various embodiments of any of the above aspects, the androgen is testosterone, dihydrotestosterone (DHT), or an analog or fragment thereof. In other embodiments, the compound is valproic acid, sodium butyrate, trichostatin A, or SAHA. In still other embodiments, the agent increases transcription or translation of tyrosine hydroxylase. In other embodiments of any of the above aspects, the compound increases dopamine synthesis. In still other embodiments of any of the above aspects, the method further involves identifying a reduction in neuronal cell death. In various embodiments of any of the above aspects, the compound is an androgen, an androgen analog, or a fragment thereof, such as testosterone, dihydrotestosterone (DHT), or an analog thereof. In other embodiments of any of the above aspects, the compound increases transcriptional or translational expression of tyrosine hydroxylase. In other embodiments of any of the above aspects, the method increases dopamine synthesis. In yet other embodiments of any of the above aspects, the method reduces neuronal apoptosis in a subject (e.g., mammal, such as a mouse or human) or increases dopamine synthesis by at least 5%, 10%, 25%, 50%, 75%, 85%, 95% or 100% in the subject. In various embodiments of the previous aspects, the compound increases transcriptional expression of tyrosine hydroxylase. In other embodiments of the previous aspects, the compound increases translational expression of tyrosine hydroxylase or increases dopamine synthesis. In other embodiments of the above aspects, the method reduces neuronal apoptosis in a subject. In yet other embodiments of the above aspects, the cell is a mammalian cell (e.g., a murine or human cell), such as a neuron (e.g., a dopaminergic neuron). In various embodiments, the method reduces cell death associated with oxidative injury in the subject. In yet other embodiments, the compound is testosterone, dihydrotestosterone (DHT), or an analog thereof. In still other embodiments of any of the above aspects, the compound increases (e.g., by at least about 5%, 10%, 25%, 50%, 75% or more) transcription or translation of tyrosine hydroxylase or increases dopamine synthesis. In still other embodiments of the above aspects, the method reduces neuronal cell death in the subject (e.g., mammal, such as a human) by at least 5%, 10%, 25%, 50%, 75% or more. In still other embodiments of any of the above aspects, the subject is male. In still other embodiments of any of the above aspects, the method increases tyrosine hydroxylase transcription or translation; increases histone acetylation; or increases Akt phosphorylation. In still other embodiments, the agent is an expression vector comprising the DJ-1 open reading frame.

### BRIEF DESCRIPTION OF THE DRAWINGS

FIGS. 1A-1F show that DJ-1 and PSF transcriptionally regulate human tyrosine hydroxylase. FIG. 1A is a Western blot showing the expression of tyrosine hydroxylase, DJ-1 and β-actin at various time points in CHP-212 cells transfected with a DJ-1 RNAi construct or with a control construct. FIG. 1B is a graph showing the relative tyrosine hydroxylase mRNA levels determined by quantitative real-time PCR (RT-PCR) in CHP-212 and SH-SY5Y cells forty-eight hours after the transfection of control (CTR) or DJ-1 RNAi (DJ-1) constructs. DJ-1 inactivation (>60%) by DJ-1 RNAi was confirmed by western blotting and RT-PCR. The tyrosine hydroxylase mRNA levels from each sample were normalized to β-actin mRNA. Values represent the mean±s.e.m. N==3 experiments *: P=0.007, **:P=0.002 relative to the control by unpaired t test. FIG. 1C is a graph showing L-Dopa production determined by the HPLC analysis in CHP-212 cells transfected with control or DJ-1-specific siRNA. Values are the mean±s.e.m; femtamole of L-Dopa/μg of protein lysate; n=12 per condition. *: P<0.01 relative to the control by one way ANOVA. FIG. 1D (left panel) is a graph showing the relative tyrosine hydroxylase mRNA levels in SH-SY5Y cells stably expressing a vector control (CTR) or the human myc-his tagged wild-type DJ-1 (DJ-1). Values are the mean±s.e.m. N=3; *: P=0.013 relative to the control by unpaired t test. FIG. 1D is a graph showing the relative tyrosine hydroxylase mRNA levels in SH-SY5Y cells stably expressing a vector control (CTR) or the human myc-his tagged wild-type DJ-1 (DJ-1). Values are the mean±s.e.m. n=3 experiments; *: p<0.05 relative to the control by unpaired t test. The side panels are Western blots indicating the expression levels of DJ-1 and β-actin in stable cells. FIG. 1E is a graph showing relative tyrosine hydroxylase mRNA levels in CHP-212 cells at 48 hours after transient transfection of a vector control (CTR) or the human wild-type PSF. Values represent the mean±s.e.m., n=2. FIG. 1F displays the results of ChIP assays showing the binding of the endogenous PSF (left panels) and DJ-1 (right panels) to the human tyrosine hydroxylase promoter in CHP-212 cells, and in the human substantia nigra pars compacta (human SN) tissue. CTR: no input DNA; Input: 0.5% of the total DNA before IP; IgG: species-matched pre-immune control antibodies for IP; PSF or DJ-1: antibodies specifically recognizing PSF or DJ-1. The results were confirmed using 3 different pairs of primers specifically amplifying the human tyrosine hydroxylase promoter sequences. Primers specific for the human GAPDH promoter were used in negative control experiments.

FIG. 2 show that DJ-1 promotes histone acetylation. FIG. 2 shows ChIP assay PCR products separated on an agarose gel. These results show that acetylated histones bound to the human tyrosine hydroxylase promoter. Various acetylated histone species from CHP-212 cells transfected with vectors expressing control or DJ-1 RNAi inserts were immunoprecipitated with specific antibodies, and amplified with primers specific for the human tyrosine hydroxylase promoter using semi-quantitative PCR. Reactions were stopped at the indicated PCR cycle an analyzed using gel electrophoresis. Input: 0.5% of input DNA before immunoprecipitation.

FIGS. 3A-3C demonstrate that the androgen receptor physically interacts with DJ-1 and binds the human tyrosine hydroxylase promoter. FIG. 3A is a Western blot showing the results of a co-immunoprecipitation using an anti-DJ-1 polyclonal antibody, which demonstrates that the endogenous androgen receptor interacts with endogenous DJ-1 present in cellular lysates prepared from a human dopaminergic neuroblastoma CHP-212 cell line. Equal amounts of pre-immune rabbit IgG were used as a control for the co-immunoprecipitation with the DJ-1 specific antibody. FIGS. 3B and 3C present the results of ChIP assays showing the binding of endogenous androgen receptor (AR) to the tyrosine hydroxylase promoter in CHP-212 cells (FIG. 4B) and human substantia nigra tissues (FIG. 3C) relative to GAPDH, which was used as a negative control. Abbreviations and their meanings follow: “WB” denotes Western blot; “CO-IP” denotes co-immunoprecipitation; “CTR” denotes control having no input DNA; “Input”: 0.5% of the total DNA before immunoprecipitation (IP); IgG: species-matched pre-immune control antibodies for IP; AR: antibody specifically recognizing AR; “GAPDH” denotes Glyseraldehyde-3-phosphate dehydrogenase; Primers specifically for the human GAPDH promoter were used in negative control experiments.

FIG. 4 includes 3 Western blots showing that the androgen receptor is required for tyrosine hydroxylase (TH) expression. In particular, this Western blot shows the temporal expression of tyrosine hydroxylase (TH), androgen receptor (AR) and β-actin at indicated time points in CHP-212 cells transfected with 100 nM of control (CTR) or AR-specific (AR) RNAi constructs. Representative Western blots of 3 independent experiments with similar results are shown.

FIGS. 5A and 5B show that dihydroxytestosterone induced tyrosine hydroxylase expression and reversed the suppression of tyrosine hydroxylase expression caused by the loss of DJ-1. FIG. 5A is a Western blot showing tyrosine hydroxylase expression levels in native CHP-212 cells treated with increasing amount of DHT (0-1000 nM). FIG. 5B shows that dihydroxytestosterone treatment reversed the inhibition of tyrosine hydroxylase expression by DJ-1 inactivation. Forty-eight hours after the transfection of control or DJ-1 specific RNAi, CHP-212 cells were treated with indicated amount of DHT daily for additional 48 hours before harvesting. The protein levels of tyrosine hydroxylase, DJ-1 and β-actin were determined by western blotting. Abbreviations and their meanings follow: “DHT” denotes dihydroxytestosterone; “TH” denotes tyrosine hydroxylase.

FIG. 6 is a Western blot showing that wild-type DJ-1 specifically induced AKT phosphorylation in human neuroblastoma SH-SY5Y cells. Equal amounts of lysates of SH-SY5Y cells stably expressing a control vector (Vec) or similar amount of myc-his tagged wild-type (Wt) or pathogenic mutant DJ-1 (homozygous M261 and heterozygous D149A) were resolved by SDS-PAGE using duplicating gels at the same time and separately probed with antibodies that specifically recognize phosphorylated or total AKT. The membranes were then re-probed with anti-DJ-1 antibody to confirm that similar levels of exogenous DJ-1 expression were present. Abbreviations and their meanings follow: “P-Akt” denotes phosphorylated Akt; “Vec” denotes vector; “WT” denotes wild-type.

FIGS. 7A-7C show that dihydroxytestosterone treatment (DHT) induced AKT phosphorylation. FIG. 7A is a Western blot showing Akt phosphorylation levels in human neuroblastoma SH-SY5Y cells treated with 10 nM DHT that were subsequently harvested at various time points between 5-90 minutes. The temporal change in AKT phosphorylation was determined by western blotting using antibodies that specifically recognize phosphorylated AKT(p-AKT) or total AKT. FIG. 7B is a Western blot of SH-SY5Y cells transiently transfected with Flag-AR that were treated with 10 nM DHT forty-eight hours after transfection, and harvested at various time points between 5-90 minutes. FIG. 7C is a graph showing a quantification of the induction of phosphorylated AKT by DHT. Three independent experiments were performed as described in FIG. 7B. The signal intensity for each protein was determined by densitometry. The levels of phosphorylated AKT was normalized to those of total AKT at the same time point and then this value is represented as the ratio to the value at the 5 minutes time point post DHT treatment. Values are the mean±s.e.m. n=3 experiments; *: P=0.0063, 0.0006 and 0.0099 (t-tests) for 15, 30 and 90 minutes of treatment, respectively. Abbreviations and their meanings follow: “Flag-AR” denotes the Flag epitope tagged androgen receptor.

FIGS. 8A and 8B are graphs showing that dihydroxytestosterone rescued dopaminergic cells from H₂O₂-induced cell toxicity. FIG. 8A is a graph that quantifies relative cell viability in SH-SY5Y cells that were pretreated with dihydroxytestosterone (DHT) (1 or 10 nM) or vehicle control (Ethanol) for twenty-four hours, then were subjected to H₂O₂ (500 μM) treatment plus dihydroxytestosterone (1 or 10 nM) or vehicle control for an additional twenty-four hours. Cell toxicity was evaluated using a 3-(4,5-dimethylthiazol-2-yl)-2,5-diphenyltetrazolium bromide (MTT) assay. Values are the mean±s.e.m. n=4 experiments in triplicates; **: P=0.0011 and 0.0083 (t-tests) for 1 or 10 nM dihydroxytestosterone treatment, respectively, relative to cells that did not receive dihydroxytestosterone treatment. FIG. 8B shows relative cell viability forty-eight hours after CHP-212 cells were transfected with control or DJ-1-specific RNAi constructs. Cells were re-plated and pretreated with 10 nM dihydroxytestosterone or vehicle control (ethanol) for twenty-four hours, followed by H₂O₂ (300 μM) treatment plus 10 nM dihydroxytestosterone or vehicle control for additional 24 hours. Cell toxicity was evaluated by MTT assay. Values are the mean±s.e.m. n=3 experiments; *: P=0.0068 (t-test).

### DETAILED DESCRIPTION OF THE INVENTION

### Definitions

By “androgen” is meant a ligand that selectively binds and activates an androgen receptor. Exemplary androgens include testosterone, dihydrotestosterone, or an analog or fragment thereof.

By “alteration” is meant a change (increase or decrease) in the expression levels of a gene or polypeptide as detected by standard art known methods such as those described above. As used herein, an alteration includes a 10% change in expression levels, preferably a 25% change, more preferably a 40% change, and most preferably a 50% or greater change in expression levels.”

By “apoptosis” is meant the process of cell death wherein a dying cell displays a set of well-characterized biochemical hallmarks that include cell membrane blebbing, cell soma shrinkage, chromatin condensation, and DNA laddering. Cells that die by apoptosis include neurons (e.g., during the course of neurodegenerative diseases such Parkinson's disease).

By “a gene required for neuronal survival or maintenance” is meant a gene encoding a polypeptide whose function is required in a neuronal cell for viability or neuronal function.

By “neurodegenerative disease” is meant any disorder characterized by excess neuronal cell death. Exemplary neurodegenerative diseases include Parkinson's disease, Huntington's disease, Alzheimer's disease, Kennedy's disease, and spinocerebellar ataxia.

By “oxidative stress” is meant any reduction in cell survival or function associated with oxidative damage.

By “tyrosine hydroxylase” is meant a polypeptide having substantial similarity to GenBank Accession No. NP_{—}954986. In one embodiment, a tyrosine hydroxylase polypeptide is encoded by GenBank Accession No. X05290.

By “analog” is meant a compound that has substantially the same function as a reference compound. An analog may or may not be structurally similar to the reference compound.

By “an effective amount” is meant the amount of a compound required to ameliorate the symptoms of a disease relative to an untreated patient. The effective amount of active compound(s) used to practice the present invention for therapeutic treatment of a neurodegenerative disease varies depending upon the manner of administration, the age, body weight, and general health of the subject. Ultimately, the attending physician or veterinarian will decide the appropriate amount and dosage regimen. Such amount is referred to as an “effective” amount.

By “increases” is meant a positive alteration of at least 10%, 15%, 25%, 50%, 75%, or 100%.

By “obtaining” as in “obtaining a compound” includes synthesizing, purchasing or otherwise acquiring the agent.

By “diagnosis” or “identifying a subject having” refers to a process of determining if an individual is afflicted with or has a genetic predispositon to develop a disease or disorder, such as a neurodegenerative disorder.

By “at risk of” is meant having a propensity to develop a disease or disorder. For example, a subject having a genetic mutation in a gene associated with a neurodegenerative disease is increased risk of developing the disease relative to a normal control subject.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

By “compound” is meant any small molecule chemical compound, antibody, nucleic acid molecule, or polypeptide, or fragments thereof.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

By “operably linked” is meant that a first polynucleotide is positioned adjacent to a second polynucleotide that directs transcription of the first polynucleotide when appropriate molecules (e.g., transcriptional activator proteins) are bound to the second polynucleotide.

By “subject” is meant a mammal, including, but not limited to, a human or non-human mammal, such as a bovine, equine, canine, ovine, or feline.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

By “reduces” is meant a negative alteration of at least 10%, 25%, 50%, 75%, or 100%.

By “reference” is meant a standard or control condition.

By “disease” is meant any condition or disorder that damages or interferes with the normal function of a cell, tissue, or organ. Examples of diseases include bacterial invasion or colonization of a host cell.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

### METHODS OF THE INVENTION

The invention generally provides therapeutic and prophylactic compositions and methods featuring androgens useful for the treatment of Parkinson's disease. The invention is based, in part, on the observation that the human androgen receptor transcriptionally activates tyrosine hydroxylase, a biosynthetic enzyme that is required for dopamine synthesis. In addition, dihydrotestosterone administration induces a dose-dependent increase in tyrosine hydroxylase expression, and reverses the transcription inhibition caused by the inactivation of DJ-1, a Parkinson's disease-related protein

### Dopamine Biosynthesis

Dopamine is a biogenic amine neurotransmitter that is derived from the amino acid tyrosine. The first step in dopamine synthesis is catalyzed by the rate-limiting enzyme tyrosine hydroxylase in a reaction requiring oxygen as a co-substrate and tetrahydrobiopterin as a cofactor to synthesize dihydroxyphenylalanine (DOPA). DOPA is subsequently decarboxylated by DOPA decarboxylase to produce dopamine.

The major dopamine-containing area of the brain is the corpus striatum, which receives major input from the substantia nigra and plays an essential role in the coordination of body movements. In Parkinson's disease the dopaminergic neurons of the substantia nigra degenerate, leading to a characteristic motor dysfunction. Although dopamine does not readily cross the blood-brain barrier, its precursor, levodopa, does. Therefore, the disease can be treated by administering levodopa together with carbidopa, a dopamine decarboxylase inhibitor, and selegiline, a monoamine oxidase inhibitor. While this treatment can alleviate some of the symptoms of Parkinson's disease, it cannot stop the degeneration of the dopaminergic neurons underlying the disorder. Inproved therapeutic methods are required.

### Androgen Receptor

As reported herein, the human androgen receptor transcriptionally activates tyrosine hydroxylase, a biosynthetic enzyme that is required for dopamine synthesis. The gene encoding the androgen receptor, alternatively known as the dihydrotestosterone receptor, is located on the X chromosome. The androgen receptor gene, which is more than 90 kb long, encodes a protein having three major functional domains: an N-terminal domain, which serves a modulatory function, a DNA-binding domain, and an androgen-binding domain. The androgen receptor is a member of the steroid hormone receptor family. In contrast to peptide hormone receptors, which span the plasma membrane and bind ligand outside the cell, steroid hormone receptors are found in the cytosol and the nucleus. Steroid hormones exert their action by passing through the plasma membrane and binding to intracellular receptors. When a steroid hormone receptor binds its ligand the receptor undergoes a conformational change that activates the receptor to recognize and bind specific nucleotide sequences termed “hormone-response elements (HREs).” Hormone-responsive elements are typically located upstream of the transcription start site and are usually composed of derivatives of palindromes with either TGACC or TGTTCT consensus sequences. The latter sequence motif is found in a variety of genes regulated by glucocorticoid, progesterone, or androgen receptors. The androgen receptor typically regulates transcription upon binding to cognate androgen-responsive elements located in the vicinity of target genes. The activity of the androgen receptor is regulated by androgens, primarily dihydrotestosterone, binding to the androgen binding domain. When ligand-receptor complexes interact with DNA they alter the transcriptional level of the associated gene.

### Testosterone

The majority of testosterone is synthesized by the Leydig cells within the testes, with some produced in the adrenal cortex. Testosterone is secreted into the plasma and in a number of target tissues, testosterone can be converted to dihydrotestosterone (DHT). DHT is the most potent of the male steroid hormones, with an activity that is 10 times that of testosterone. Because of its relatively lower potency, testosterone is sometimes considered to be a prohonnone.

### Loss of Function in DJ-1 is Linked to Parkinson's Disease

Mutations in genes including α-synuclein, parkin, PINK-1, DJ-1, and LRRK have been definitively linked to familial Parkinson's disease. Genetic evidence suggests the presence of potential common pathways affected by the Parkinson's disease -related proteins¹⁻³. As in parkin and PINK-1, deletions or point mutations in DJ-1 cause autosomal recessive early-onset Parkinson's di sease³⁻⁵. Before DJ-1 was linked to Parkinson's disease, studies revealed that DJ-1 possesses oncogenic potential and affects spermatogenesis⁶⁻⁸. In addition, DJ-1 regulates androgen receptor-mediated transcription by interacting with transcriptional repressor PIASxa and DJBP in the testis^{9,10}. Biochemically, DJ-1 adopts a more acidic form upon oxidative stress, suggesting its potential roles in stress response^{11,12}. The crystal structure of DJ-1 has been solved and indicates that DJ-1 exists as a dimer and structurally resembles a bacterial cysteine protease¹³⁻¹⁶. Cell biology studies indicate that the homozygous mutations, L166P and M26I, render DJ-1 unstable¹⁷⁻²¹, while heterozygous pathogenic DJ-1 mutations, such as D149A and A104T, attenuated the normal DJ-1 functions^{18,19,22}. The sequence of a DJ-1 polypeptide is provided at NP_{—}009193. A nucleic acid sequence of DJ-1 is provided at AB045294.

The neuroprotective and anti-apoptotic activities of DJ-1 have been clearly demonstrated, although multiple cellular mechanisms, including the regulation of oxidative stress response^{12,23-25}, survival pathway^{26,27}, signal transduction^{26,27} and transcription¹⁸, have been proposed. In vitro, DJ-1 has been shown to be a cysteine protease²⁸ and a molecular chaperone preventing protein aggregation²⁹. Furthermore, DJ-1-deficient mice have been established by multiple groups³⁰⁻³². Although these mice do not reproduce typical symptoms observed in Parkinson's disease patients, they demonstrate moderate defects in the nigralstiatal pathway, including abnormal dopamine uptake³⁰, age-dependent locomotor deficits³² or hypersensitivity to the mitochondrial toxin MPTP³¹. Recently, Drosophila lacking DJ-1 expression have been shown to be vulnerable to oxidative stress³³⁻³⁵. These studies support the role of DJ-1 in Parkinson's disease pathogenesis. The results reported herein, indicate a direct link between DJ-1 and the nigralstiatal pathway by demonstrating that human tyrosine hydroxylase(TH), the rate-limiting enzyme for dopamine synthesis, is transcriptionally upregulated by DJ-1. Therefore, loss of DJ-1 functions will impact both neuronal survival and dopamine production.

Screens for Compounds that Enhance Cell Survival

Compounds that enhance the transcriptional activation of tyrosine hydroxylase by an androgen may also enhance the survival of neuronal cells at risk of undergoing apoptosis. If desired, compounds that modulate transcriptional activity of a gene of interest (e.g., tyrosine hydroxylase) are tested for efficacy in reducing cell death in a cell (e.g., a dopaminergic neuronal cell) at risk thereof. In one example, a candidate compound in combination with an androgen is added to the culture medium of cells (e.g., neuronal cultures) prior to, concurrent with, or following the addition of a proapoptotic agent. Cell survival is then measured using standard methods. The level of apoptosis in the presence of the candidate compound is compared to the level measured in a control culture medium lacking the candidate compound. A compound that promotes an increase in cell survival, a reduction in apoptosis, or an increase in cell proliferation in combination with an androgen is considered useful in the invention; such a candidate compound may be used, for example, as a therapeutic in combination with an androgen to prevent, delay, ameliorate, stabilize, or treat a disease or disorder characterized by excess cell death (e.g., a neurodegenerative disorder). Alternatively, the combination of the candidate compound and the androgen promotes the survival of a neuronal cell at risk of cell death. Such therapeutic compounds and combinations are useful in vivo as well as ex vivo.

Compounds isolated by this method (or any other appropriate method) may, if desired, be further purified (e.g., by high performance liquid chromatography). In addition, these candidate compounds may be tested for their ability to modulate transcriptional activity in a neuronal cell, to reduce cell death, or to promote cell survival. Compounds isolated by this approach may be used, for example, as therapeutics to treat a neurodegenerative disease in a subject.

One skilled in the art appreciates that the effects of a candidate compound in combination with an androgen on transcriptional activation or cell survival are typically compared to transcriptional activation or cell survival in the absence of the candidate compound.

Compounds that increase transcriptional activation or cell survival include organic molecules, peptides, peptide mimetics, polypeptides, and nucleic acids. Each of the sequences listed herein may also be used in the discovery and development of a therapeutic compound for the treatment of a neurodegenerative disease. The encoded protein, upon expression, can be used as a target for the screening of drugs. Additionally, the DNA sequences encoding the amino terminal regions of the encoded protein or Shine-Delgarno or other translation facilitating sequences of the respective mRNA can be used to construct sequences that promote the expression of the coding sequence of interest. Such sequences may be isolated by standard techniques (Ausubel et al., supra). Small molecules of the invention preferably have a molecular weight below 2,000 daltons, more preferably between 300 and 1,000 daltons, and most preferably between 400 and 700 daltons. It is preferred that these small molecules are organic molecules.

The invention also includes novel compounds identified by the above-described screening assays. Optionally, such compounds are characterized in one or more appropriate animal models to determine the efficacy of the compound for the treatment of a neurodegenerative disease. Desirably, characterization in an animal model can also be used to determine the toxicity, side effects, or mechanism of action of treatment with such a compound. Furthermore, novel compounds identified in any of the above-described screening assays may be used for the treatment of a neurodegenerative disease in a subject. Such compounds are useful alone or in combination with other conventional therapies known in the art.

### Cells for Use in Screens

In one embodiment, the screens described herein are carried out in dopaminergic cells having neuronal characteristics. Such cells are known in the art and include, for example, BE(2)-M17 neuroblastoma cells (Martin et al., J. Neurochem. 2003 November; 87(3):620-30), Cath.a-differentiated (CAD) cells (Arboleda et al., J Mol. Neurosci. 2005; 27(1):65-78), CSM14.1 (Haas et al., J. Anat. 2002 July; 201(1):61-9), MN9D (Chen et al., Neurobiol Dis. 2005 August; 19(3):419-26), N27 cells (Kaul et al., J Biol. Chem. 2005 Aug. 5; 280(31):28721-30), PC12 (Gonnan et al., Biochem Biophys Res Commun. 2005 Feb. 18; 327(3):801-10), SN4741 (Nair et al., Biochem J. 2003 Jul. 1; 373(Pt 1):25-32), CHP-212, SH-SY5Y, and SK-N-BE.

### Test Compounds and Extracts

In general, compounds capable of modulating transcriptional activation or cell survival are identified from large libraries of both natural product or synthetic (or semi-synthetic) extracts or chemical libraries or from polypeptide or nucleic acid libraries, according to methods known in the art. Those skilled in the field of drug discovery and development will understand that the precise source of test extracts or compounds is not critical to the screening procedure(s) of the invention. Compounds used in screens may include known compounds (for example, known therapeutics used for other diseases or disorders). Alternatively, virtually any number of unknown chemical extracts or compounds can be screened using the methods described herein. Examples of such extracts or compounds include, but are not limited to, plant-, fungal-, prokaryotic- or animal-based extracts, fermentation broths, and synthetic compounds, as well as modification of existing compounds.

Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). Alternatively, chemical compounds to be used as candidate compounds can be synthesized from readily available starting materials using standard synthetic techniques and methodologies known to those of ordinary skill in the art. Synthetic chemistry transformations and protecting group methodologies (protection and deprotection) useful in synthesizing the compounds identified by the methods described herein are known in the art and include, for example, those such as described in R. Larock, Comprehensive Organic Transformations, VCH Publishers (1989); T. W. Greene and P. G. M. Wuts, Protective Groups in Organic Synthesis, 2nd ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

Alternatively, libraries of natural compounds in the form of bacterial, fungal, plant, and animal extracts are commercially available from a number of sources, including Biotics (Sussex, UK), Xenova (Slough, UK), Harbor Branch Oceanographic Institute (Ft. Pierce, Fla.), and PharmaMar, U.S.A. (Cambridge, Mass.). In addition, natural and synthetically produced libraries are produced, if desired, according to methods known in the art, e.g., by standard extraction and fractionation methods. Examples of methods for the synthesis of molecular libraries can be found in the art, for example in: DeWitt et al., Proc. Natl. Acad. Sci. U.S.A. 90:6909, 1993; Erb et al., Proc. Natl. Acad. Sci. USA 91:11422, 1994; Zuckermann et al., J. Med. Chem. 37:2678, 1994; Cho et al., Science 261:1303, 1993; Carrell et al., Angew. Chem. Int. Ed. Engl. 33:2059, 1994; Carell et al., Angew. Chem. Int. Ed. Engl. 33:2061, 1994; and Gallop et al., J. Med. Chem. 37:1233, 1994. Furthermore, if desired, any library or compound is readily modified using standard chemical, physical, or biochemical methods.

Libraries of compounds may be presented in solution (e.g., Houghten, Biotechniques 13:412-421, 1992), or on beads (Lam, Nature 354:82-84, 1991), chips (Fodor, Nature 364:555-556, 1993), bacteria (Ladner, U.S. Pat. No. 5,223,409), spores (Ladner U.S. Pat. No. 5,223,409), plasmids (Cull et al., Proc Natl Acad Sci USA 89:1865-1869, 1992) or on phage (Scott and Smith, Science 249:386-390, 1990; Devlin, Science 249:404-406, 1990; Cwirla et al. Proc. Natl. Acad. Sci. 87:6378-6382, 1990; Felici, J. Mol. Biol. 222:301-310, 1991; Ladner supra.).

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their activity should be employed whenever possible.

When a crude extract of interest is identified, further fractionation of the positive lead extract is necessary to isolate chemical constituents responsible for the observed effect. Thus, the goal of the extraction, fractionation, and purification process is the careful characterization and identification of a chemical entity within the crude extract that alters the transcriptional activity of a gene associated with a neurodegenerative disease. Methods of fractionation and purification of such heterogenous extracts are known in the art. If desired, compounds shown to be useful as therapeutics for the treatment of a neurodegenerative disease are chemically modified according to methods known in the art.

### Pharmaceutical Therapeutics

The invention provides androgens and androgen derivatives, as well as compounds identified in the above-identified screens, for the treatment of a neurodegenerative disease. Accordingly, a chemical entity discovered to have medicinal value using the methods described herein is useful as a drug or as information for structural modification of existing compounds, e.g., by rational drug design. For therapeutic uses, the compositions or agents identified using the methods disclosed herein may be administered systemically, for example, formulated in a pharmaceutically-acceptable carrier. Preferable routes of administration include, for example, subcutaneous, intravenous, interperitoneally, intramuscular, or intradermal injections that provide continuous, sustained levels of the drug in the patient. Treatment of human patients or other animals will be carried out using a therapeutically effective amount of a neurodegenerative disease therapeutic in a physiologically-acceptable carrier. Suitable carriers and their formulation are described, for example, in Remington's Pharmaceutical Sciences by E. W. Martin. The amount of the therapeutic agent to be administered varies depending upon the manner of administration, the age and body weight of the patient, and the clinical symptoms of the neurodegenerative disease. Generally, amounts will be in the range of those used for other agents used in the treatment of a neurodegenerative disease, although in certain instances lower amounts will be needed because of the increased specificity of the compound. A compound is administered at a dosage that controls the clinical or physiological symptoms of a neurodegenerative disease as determined by a diagnostic method known to one skilled in the art, or using any that assay that measures the transcriptional activation of a gene associated with a neurodegenerative disease.

### Formulation of Pharmaceutical Compositions

The administration of an androgen or analog thereof for the treatment of a neurodegenerative disease may be by any suitable means that results in a concentration of the therapeutic that, combined with other components, is effective in ameliorating, reducing, or stabilizing the neurodegenerative disease or a symptom thereof. In one embodiment, administration of the androgen enhances tyrosine hydroxylase expression. Accordingly, androgens, androgen analogs, and fragments thereof are useful in the methods of the invention. In one embodiment, testosterone or dihydrotestosterone (DHT) is administered to a subject for the prevention or treatment of Parkinson's disease.

Methods of administering androgens, such as testosterone or DHT, are known in the art. While ingested testosterone is readily absorbed into the circulation, the hormone is rapidly catabolized by the liver, and thus does not reach therapeutic serum levels following oral administration. Thus, preferred methods for testosterone delivery are typically designed to bypass hepatic catabolism. In one embodiment, an esterified testosterone, such as testosterone enanthate (heptanoate) or cypionate (cyclopentylpropionate) is dissolved in oil and administered intramuscularly every two to four weeks. Testosterone undecanoate in oil may be ingested orally or injected. Oral administration of testosterone undecanoate in oil is absorbed into the lymphatic circulation thus bypassing initial hepatic catabolism. Other oral formulations of testosterone include testosterone derivatives such as 17β-esters, 7α-methyl, 17α-alkyl or methyl, 19-normethyl and D-homoandrogens Handelsman, “Testosterone and Other Androgens: Physiology, Pharmacology, and Therapeutic Use,” in Endocrinology—Volume 3, Ed's DeGroot et al. (1995), pp. 2351-2361. Other testosterone derivatives include, but are not limited to, testosterone substituted at the Cl position with methyl, e.g., methenolone and mesterolone. In some embodiments, testosterone is administered in a transdermal preparation. Transdermal preparations include TESTODERM®, TESTODERM®, and ANDRODERM®. For some applications testosterone is administered as an injectable formulation, such as DEPO-TESTOSTERONE® (testosterone cypionate), and DELATESTRYL BTG® (testosterone enanthate), or as a gel, for example, ANDROGEL®. Other testosterone formulations are provided in U.S. Pat. No. 6,319,913; or in U.S. Patent Publication No. 20030216328.

Testosterone may also be administered as a pharmaceutically acceptable salt; testosterone salts include, but are not limited to acetate, enanthate, cypionate, isobutyrate, propionate, and undecanoate esters, cyproterone acetate, danazol, finasteride, fluoxymesterone, methyltestosterone, nandrolone decanoate, nandrolone phenpropionate, oxandrolone, oxymetholone, stanozolol, and testolactone. Androgen analogs useful in the methods of the invention include, but are not limited to danazol (Danocrine®), fluoxymesterone (Halotestin®), 17-α methyl testosterone, nandrolone derivatives, 5-α-dihydrotestosterone, and 7-α methyl-19-nortestosterone. Other methods for the administration of testosterone are known in the art, and are described, for example, in U.S. Patent Publications Nos: 20050118242, 20040235808, and 20040220154.

The invention provides for the therapeutic administration of an androgen by any means known in the art. The compound may be contained in any appropriate amount in any suitable carrier substance, and is generally present in an amount of 1-95% by weight of the total weight of the composition. The composition may be provided in a dosage form that is suitable for parenteral (e.g., subcutaneously, intravenously, intramuscularly, or intraperitoneally) administration route. The pharmaceutical compositions may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York). Suitable formulations include forms for oral administration, depot formulations, formulations for delivery by a patch, such as a scrotal patch, semisolid dosage forms to be topically or transdermally delivered.

Pharmaceutical compositions according to the invention may be formulated to release the active compound substantially immediately upon administration or at any predetermined time or time period after administration. The latter types of compositions are generally known as controlled release formulations, which include (i) formulations that create a substantially constant concentration of the drug within the body over an extended period of time; (ii) formulations that after a predetermined lag time create a substantially constant concentration of the drug within the body over an extended period of time; (iii) formulations that sustain action during a predetermined time period by maintaining a relatively, constant, effective level in the body with concomitant minimization of undesirable side effects associated with fluctuations in the plasma level of the active substance (sawtooth kinetic pattern); (iv) formulations that localize action by, e.g., spatial placement of a controlled release composition adjacent to or in the central nervous system or cerebrospinal fluid; (v) formulations that allow for convenient dosing, such that doses are administered, for example, once every one or two weeks; and (vi) formulations that target a neurodegenerative disease by using carriers or chemical derivatives to deliver the therapeutic agent to a particular cell type (e.g., neuronal cell at risk of cell death) whose function is perturbed in the neurodegenerative disease. For some applications, controlled release formulations obviate the need for frequent dosing during the day to sustain the plasma level at a therapeutic level.

Any of a number of strategies can be pursued in order to obtain controlled release in which the rate of release outweighs the rate of metabolism of the compound in question. In one example, controlled release is obtained by appropriate selection of various formulation parameters and ingredients, including, e.g., various types of controlled release compositions and coatings. Thus, the therapeutic is formulated with appropriate excipients into a pharmaceutical composition that, upon administration, releases the therapeutic in a controlled manner. Examples include single or multiple unit tablet or capsule compositions, oil solutions, suspensions, emulsions, microcapsules, microspheres, molecular complexes, nanoparticles, patches, and liposomes.

### Parenteral Compositions

The pharmaceutical composition may be administered parenterally by injection, infusion or implantation (subcutaneous, intravenous, intramuscular, intraperitoneal, or the like) in dosage forms, formulations, or via suitable delivery devices or implants containing conventional, non-toxic pharmaceutically acceptable carriers and adjuvants. The formulation and preparation of such compositions are well known to those skilled in the art of pharmaceutical formulation. Formulations can be found in Remington: The Science and Practice of Pharmacy, supra.

Compositions for parenteral use may be provided in unit dosage forms (e.g., in single-dose ampoules), or in vials containing several doses and in which a suitable preservative may be added (see below). The composition may be in the form of a solution, a suspension, an emulsion, an infusion device, or a delivery device for implantation, or it may be presented as a dry powder to be reconstituted with water or another suitable vehicle before use. Apart from the active therapeutic (s), the composition may include suitable parenterally acceptable carriers and/or excipients. The active therapeutic (s) may be incorporated into microspheres, microcapsules, nanoparticles, liposomes, or the like for controlled release. Furthermore, the composition may include suspending, solubilizing, stabilizing, pH-adjusting agents, tonicity adjusting agents, and/or dispersing, agents.

As indicated above, the pharmaceutical compositions according to the invention may be in the form suitable for sterile injection. To prepare such a composition, the suitable active therapeutic(s) are dissolved or suspended in a parenterally acceptable liquid vehicle.

### Controlled Release Parenteral Compositions

Controlled release parenteral compositions may be in the form of suspensions, microspheres, microcapsules, magnetic microspheres, oil solutions, oil suspensions, or emulsions. Alternatively, the active drug may be incorporated in biocompatible carriers, liposomes, nanoparticles, implants, or infusion devices. Materials for use in the preparation of microspheres and/or microcapsules are, e.g., biodegradable/bioerodible polymers such as polygalactin, poly-(isobutyl cyanoacrylate), poly(2-hydroxyethyl-L-glutam-nine) and, poly(lactic acid). Biocompatible carriers that may be used when formulating a controlled release parenteral formulation are carbohydrates (e.g., dextrans), proteins (e.g., albumin), lipoproteins, or antibodies. Materials for use in implants can be non-biodegradable (e.g., polydimethyl siloxane) or biodegradable (e.g., poly(caprolactone), poly(lactic acid), poly(glycolic acid) or poly(ortho esters) or combinations thereof).

### Solid Dosage Forms for Oral Use

Formulations for oral use include tablets containing an active ingredient(s) in a mixture with non-toxic pharmaceutically acceptable excipients. Such formulations are known to the skilled artisan. Excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscannellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

The tablets may be uncoated or they may be coated by known techniques, optionally to delay disintegration and absorption in the gastrointestinal tract and thereby providing a sustained action over a longer period. The coating may be adapted to release the active drug in a predetermined pattern (e.g., in order to achieve a controlled release formulation) or it may be adapted not to release the active drug until after passage of the stomach (enteric coating). The coating may be a sugar coating, a film coating (e.g., based on hydroxypropyl methylcellulose, methylcellulose, methyl hydroxyethylcellulose, hydroxypropylcellulose, carboxymethylcellulose, acrylate copolymers, polyethylene glycols and/or polyvinylpyrrolidone), or an enteric coating (e.g., based on methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose). Furthermore, a time delay material such as, e.g., glyceryl monostearate or glyceryl distearate may be employed.

The solid tablet compositions may include a coating adapted to protect the composition from unwanted chemical changes, (e.g., chemical degradation prior to the release of the active neurodegenerative disease therapeutic substance). The coating may be applied on the solid dosage form in a similar manner as that described in Encyclopedia of Pharmaceutical Technology, supra.

At least two active neurodegenerative disease therapeutics may be mixed together in the tablet, or may be partitioned. In one example, the first active therapeutic is contained on the inside of the tablet, and the second active therapeutic is on the outside, such that a substantial portion of the second active therapeutic is released prior to the release of the first active therapeutic.

Formulations for oral use may also be presented as chewable tablets, or as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders and granulates may be prepared using the ingredients mentioned above under tablets and capsules in a conventional manner using, e.g., a mixer, a fluid bed apparatus or a spray drying equipment.

### Controlled Release Oral Dosage Forms

Controlled release compositions for oral use may be constructed to release the active neurodegenerative disease therapeutic by controlling the dissolution and/or the diffusion of the active substance. Dissolution or diffusion controlled release can be achieved by appropriate coating of a tablet, capsule, pellet, or granulate formulation of compounds, or by incorporating the compound into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

A controlled release composition containing one or more therapeutic compounds may also be in the form of a buoyant tablet or capsule (i.e., a tablet or capsule that, upon oral administration, floats on top of the gastric content for a certain period of time). A buoyant tablet formulation of the compound(s) can be prepared by granulating a mixture of the compound(s) with excipients and 20-75% w/w of hydrocolloids, such as hydroxyethylcellulose, hydroxypropylcellulose, or hydroxypropylmethylcellulose. The obtained granules can then be compressed into tablets. On contact with the gastric juice, the tablet forms a substantially water-impermeable gel barrier around its surface. This gel barrier takes part in maintaining a density of less than one, thereby allowing the tablet to remain buoyant in the gastric juice.

### Topical Administration Forms

Dosage forms for the semisolid topical administration of a mammalian androgen of this invention include ointments, pastes, creams, lotions, and gels. The dosage forms may be formulated with mucoadhesive polymers for sustained release of active ingredients at the area of application to the skin. The active compound may be mixed under sterile conditions with a pharmaceutically acceptable carrier, and with any preservatives, buffers, or propellants, which may be required. Such topical preparations can be prepared by combining the compound of interest with conventional pharmaceutical diluents and carriers commonly used in topical liquid, cream, and gel formulations.

Ointments and creams may, for example, be formulated with an aqueous or oily base with the addition of suitable thickening and/or gelling agents. Such bases may include water and/or an oil (e.g., liquid paraffin, vegetable oil, such as peanut oil or castor oil). Thickening agents that may be used according to the nature of the base include soft paraffin, aluminum stearate, cetostearyl alcohol, propylene glycol, polyethylene glycols, woolfat, hydrogenated lanolin, beeswax, and the like.

Lotions may be formulated with an aqueous or oily base and, in general, also include one or more of the following: stabilizing agents, emulsifying agents, dispersing agents, suspending agents, thickening agents, coloring agents, perfumes, and the like. The ointments, pastes, creams and gels also may contain excipients, including, but not limited to, animal and vegetable fats, oils, waxes, paraffins, starch, tragacanth, cellulose derivatives, polyethylene glycols, silicones, bentonites, silicic acid, talc and zinc oxide, or mixtures thereof.

Suitable excipients, depending on the hormone, include petrolatum, lanolin, methylcellulose, sodium carboxymethylcellulose, hydroxpropylcellulose, sodium alginate, carbomers, glycerin, glycols, oils, glycerol, benzoates, parabens and surfactants. It will be apparent to those of skill in the art that the solubility of a particular compound will, in part, determine how the compound is formulated. An aqueous gel formulation is suitable for water soluble compounds. Where a compound is insoluble in water at the concentrations required for activity, a cream or ointment preparation will typically be preferable. In this case, oil phase, aqueous/organic phase and surfactant may be required to prepare the formulations. Thus, based on the solubility and excipient-active interaction information, the dosage forms can be designed and excipients can be chosen to formulate the prototype preparations.

The topical pharmaceutical compositions can also include one or more preservatives or bacteriostatic agents, e.g., methyl hydroxybenzoate, propyl hydroxybenzoate, chlorocresol, benzalkonium chlorides, and the like. The topical pharmaceutical compositions also can contain other active ingredients including, but not limited to, antimicrobial agents, particularly antibiotics, anesthetics, analgesics, and antipruritic agents.

### Dosage

Human dosage amounts can initially be determined by extrapolating from the amount of compound used in mice, as a skilled artisan recognizes it is routine in the art to modify the dosage for humans compared to animal models. In certain embodiments it is envisioned that the dosage may vary from between about 1 mg compound/Kg body weight to about 5000 mg compound/Kg body weight; or from about 5 mg/Kg body weight to about 4000 mg/Kg body weight or from about 10 mg/Kg body weight to about 3000 mg/Kg body weight; or from about 50 mg/Kg body weight to about 2000 mg/Kg body weight; or from about 100 mg/Kg body weight to about 1000 mg/Kg body weight; or from about 150 mg/Kg body weight to about 500 mg/Kg body weight. In other embodiments this dose may be about 1, 5, 10, 25, 50, 75, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1600, 1700, 1800, 1900, 2000, 2500, 3000, 3500, 4000, 4500, 5000 mg/Kg body weight. In other embodiments, it is envisaged that higher does may be used, such doses may be in the range of about 5 mg compound/Kg body to about 20 mg compound/Kg body. In other embodiments the doses may be about 8, 10, 12, 14, 16 or 18 mg/Kg body weight. Of course, this dosage amount may be adjusted upward or downward, as is routinely done in such treatment protocols, depending on the results of the initial clinical trials and the needs of a particular patient.

### Therapeutic Methods

The present invention provides methods of treating a neurodegenerative disease or symptoms thereof (e.g., cytotoxicity) by modulating the transcriptional activity of a gene required for neuronal survival or maintenance. The methods comprise administering a therapeutically effective amount of a pharmaceutical composition comprising a compound that modulates transcriptional activity using the methods described herein to a subject (e.g., a mammal such as a human). Thus, one embodiment is a method of treating a subject suffering from or susceptible to a neurodegenerative disease or symptom thereof. The method includes the step of administering to the subject a therapeutic amount of an amount of a compound herein sufficient to treat the disease or symptom thereof, under conditions such that the disease is treated.

The methods herein include administering to the subject (including a subject identified as in need of such treatment) an effective amount of a compound described herein, or a composition described herein to produce such effect. Identifying a subject in need of such treatment can be in the judgment of a subject or a health care professional and can be subjective (e.g. opinion) or objective (e.g. measurable by a test or diagnostic method).

The therapeutic methods of the invention, which include prophylactic treatment, in general comprise administration of a therapeutically effective amount of the compounds herein, such as a compound of the formulae herein to a subject (e.g., animal, human) in need thereof, including a mammal, particularly a human. Such treatment will be suitably administered to subjects, particularly humans, suffering from, having, susceptible to, or at risk for a neurodegenerative disease or symptom thereof. Determination of those subjects “at risk” can be made by any objective or subjective determination by a diagnostic test or opinion of a subject or health care provider (e.g., genetic test, enzyme or protein marker, Marker (as defined herein), family history, and the like). The compounds herein may be also used in the treatment of any other disorders in which transcriptional activity may be implicated.

In one embodiment, the invention provides a method of monitoring treatment progress. The method includes the step of determining a level of diagnostic marker (Marker) (e.g., any target delineated herein modulated by a compound herein, a protein or indicator thereof, etc.) or diagnostic measurement (e.g., screen, assay) in a subject suffering from or susceptible to a disorder or symptoms thereof associated with a neurodegenerative disease, in which the subject has been administered a therapeutic amount of a compound herein sufficient to treat the disease or symptoms thereof. The level of Marker determined in the method can be compared to known levels of Marker in either healthy normal controls or in other afflicted patients to establish the subject's disease status. In preferred embodiments, a second level of Marker in the subject is determined at a time point later than the determination of the first level, and the two levels are compared to monitor the course of disease or the efficacy of the therapy. In certain preferred embodiments, a pre-treatment level of Marker in the subject is determined prior to beginning treatment according to this invention; this pre-treatment level of Marker can then be compared to the level of Marker in the subject after the treatment commences, to determine the efficacy of the treatment.

The following examples are provided to illustrate the invention, not to limit it. Those skilled in the art will understand that the specific constructions provided below may be changed in numerous ways, consistent with the above described invention while retaining the critical properties of the compounds or combinations thereof.

### Kits

The invention provides kits for the treatment or prevention of a neuronal degenerative disorder. In one embodiment, the kit includes a therapeutic or prophylactic composition containing an effective amount of an androgen in unit dosage form. In some embodiments, the kit comprises a sterile container which contains a therapeutic or prophylactic compound; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

If desired an androgen of the invention is provided together with instructions for administering it to a subject having or at risk of developing a neurodegenerative disorder. The instructions will generally include information about the use of the composition for the treatment or prevention of the neurodegenerative disorder. In other embodiments, the instructions include at least one of the following: description of the compound; dosage schedule and administration for treatment or prevention of a neurodegenerative disorder or symptoms thereof; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

### Combination Therapies

Optionally, an androgen having therapeutic or prophylactic efficacy may be administered in combination with any other standard therapy for the treatment of a neurodegenerative disease; such methods are known to the skilled artisan and described in Remington's Pharmaceutical Sciences by E. W. Martin. If desired, androgens of the invention may be administered alone or in combination with a conventional therapeutic useful for the treatment of a neurodegenerative disease disease. Therapeutics useful for the treatment of Parkinson's disease include, but are not limited to, deprenyl, amantadine or anticholinergic medications, levodopa, carbidopa, entacapone, pramipexole, rasagiline, antihistamines, antidepressants, dopamine agonists, monoamine oxidase inhibitors (MAOIs), and others. Therapeutics useful for the treatment of Huntington's disease include, but are not limited to, dopamine blockers (e.g., haloperidol or phenothiazine), reserpine, tetrabenazine and amantadine and co-enzyme Q10.

### EXAMPLES

### Example 1

### DJ-1 and PSF Transcriptionally Regulates the Human Tyrosine Hydroxylase Promoter

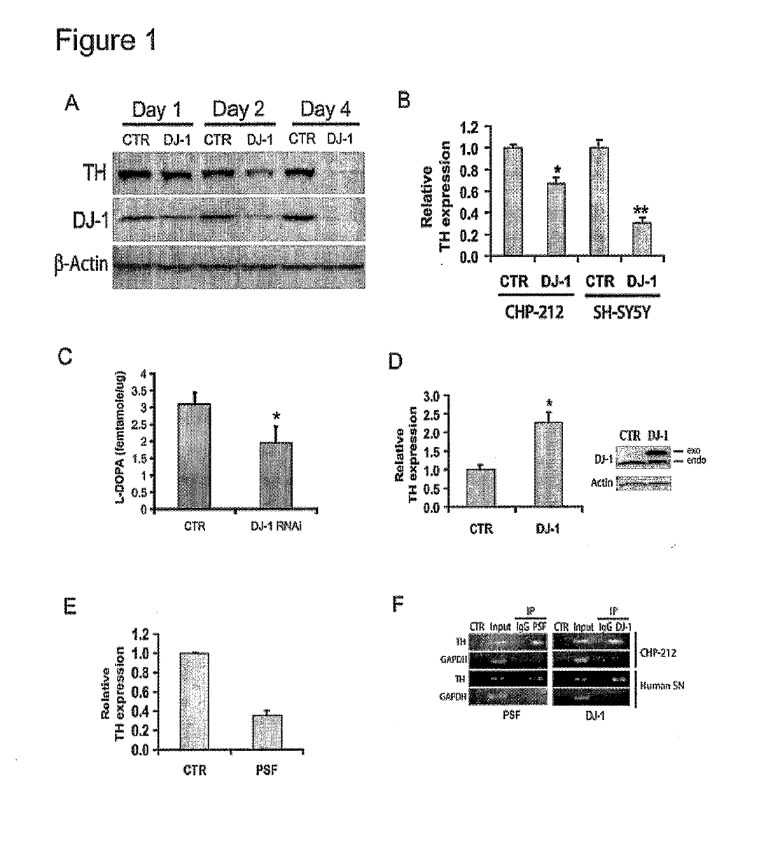

DJ-1 is a transcriptional co-activator. To determine whether DJ-1 regulated the expression of genes involved in dopaminergic neurotransmission, such as tyrosine hydroxylase, the rate-limiting enzyme that converts tyrosine to the dopamine precursor L-Dopa, DJ-1-specific siRNA constructs were used to inhibit the synthesis of endogenous DJ-1 in two human dopaminergic neuroblastoma cell lines, CHP-212 and SH-SY5Y cells. Expression of the DJ-1-specific siRNA mimicked the loss-of-function effects seen in Parkinson's disease patients with DJ-1 mutations. The protein levels of tyrosine hydroxylase and DJ-1 showed time-dependent decreases in CHP-212 cells transfected with DJ-1-specific siRNA (FIG. 1A). Four days after DJ-1 siRNA transfection, tyrosine hydroxylase protein expression was reduced by 90% (FIG. 1A). Quantitative real-time PCR results indicated that DJ-1 inactivation by siRNA significantly decreased the tyrosine hydroxylase mRNA levels in both CHP-212 and SH-SY5Y cells as determined by quantitative real-time PCR (FIG. 1B). In addition, the reduction in the tyrosine hydroxylase expression following siRNA knockdown of DJ-1 decreased the tyrosine hydroxylase activity by almost 40% in CHP-212 cells, as determined by the production of L-Dopa using HPLC (FIG. 1C). Consistent with these observations, the tyrosine hydroxylase mRNA expression was increased by more than 100% in SH-SY5Y cells stably expressing the human wild-type DJ-1 (FIG. 1D).

DJ-1 interacts with and blocks the functions of a transcriptional repressor PSF in human dopaminergic cells. As in SH-SY5Y cells, PSF specifically interacted with DJ-1 in untransfected CHP-212 cells. Therefore, to determine whether PSF repressed tyrosine hydroxylase transcription, wild-type PSF was transiently expressed in CHP-212 cells. The expression of wild-type PSF inhibited human tyrosine hydroxylase mRNA expression in CHP-212 cells (FIG. 1E). To confirm the transcriptional regulation of the human tyrosine hydroxylase promoter by both DJ-1 and PSF, chromatin immunoprecipitation (ChIP) assays were performed to assess the physical interactions between these two transcriptional regulators and the tyrosine hydroxylase promoter in vitro and in vivo. The DNA co-immunoprecipitated with either a monoclonal anti-PSF or a polyclonal anti-DJ-1 antibody using the lysates from CHP-212 cells or human substantia nigra pars compacta (SNpc) tissues were amplified by primers that specifically recognize the human tyrosine hydroxylase promoter, but not by primers recognizing the human GAPDH promoter (FIG. 1F). Taken together, these results strongly demonstrated that DJ-1 activates the human tyrosine hydroxylase expression and regulates dopamine synthesis.

### Example 2

### DJ-1 Promotes Histone Acetylation

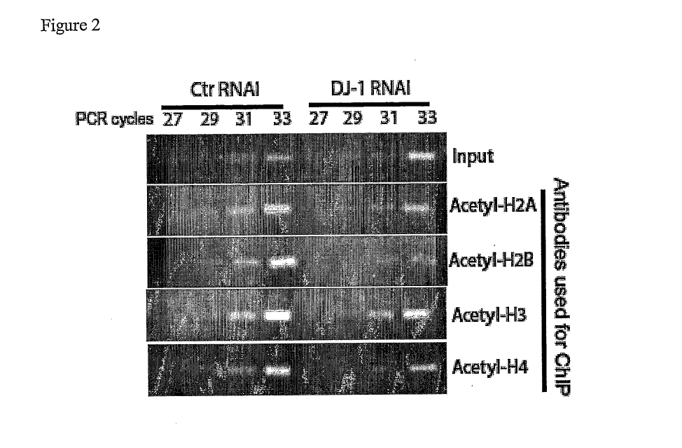

To explore the mechanism whereby DJ-1 upregulates the human tyrosine hydroxylase promoter, a potential role of DJ-1 in histone acetylation was examined, particularly the histones associated with the human tyrosine hydroxylase promoter. Increased acetylation of nucleasomal histones is known to promote gene expression. CHP-212 cells were transfected with DJ-1-specific or control siRNAs (FIG. 2). ChIP assays were then performed with antibodies that specifically recognize acetylated histones, and amplify the tyrosine hydroxylase promoter sequences using semi-quantitative PCR. Consistent with the concurrent inhibition of tyrosine hydroxylase (not shown), DJ-1 inactivation resulted in decreased acetylation of the tyrosine hydroxylase promoter-bound histones (FIG. 2).

### Example 3

### Androgen Receptor Interacts with DJ-1 and Transcriptionally Activates the Human TH Promoter

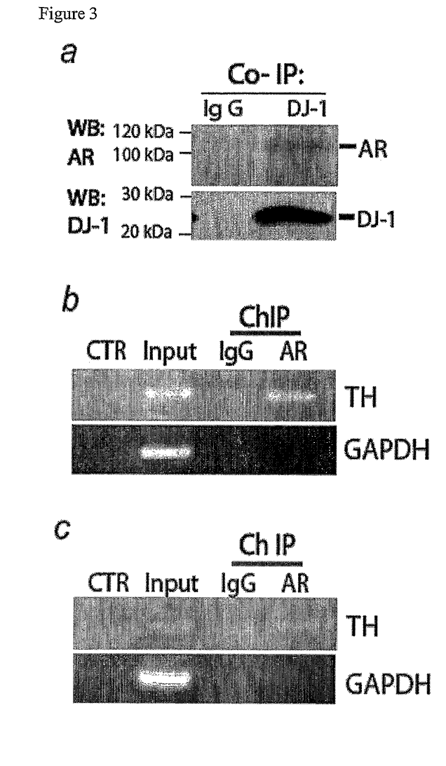

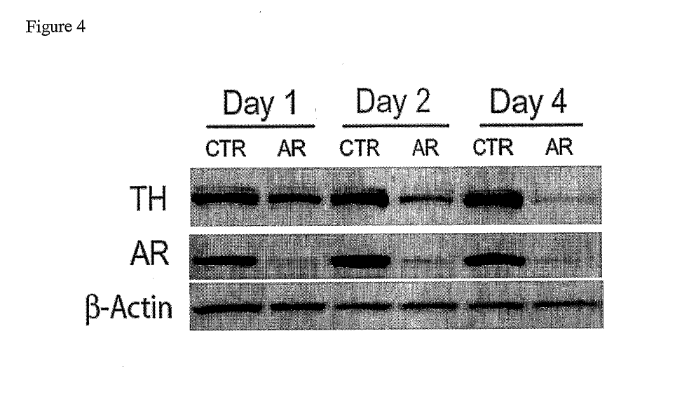

DJ-1 transcriptionally activates the human tyrosine hydroxylase promoter in a human dopaminergic neuroblastoma cell line (CHP212) by blocking the repression by PSF⁴⁹. Given that DJ-1 acts as a positive regulator of androgen receptor (AR)^{50,51}, and that PSF binds one of the activation domains of androgen receptor⁵², androgen receptor may regulate the expression of the human tyrosine hydroxylase promoter and DJ-1 may act as a co-activator. The expression of androgen receptor and the interaction between androgen receptor and DJ-1 was examined in native CHP212 cells. Consistent with ChIP results described above, co-immunoprecipitation experiments also indicated that DJ-1 interacted with endogenous androgen receptor in native CHP-212 cells (FIG. 3A). In addition, like DJ-1, the androgen receptor specifically bound the human tyrosine hydroxylase promoter not only in CHP-212 cells, but also in human substantia nigral tissues as assayed in chromatin immunoprecipitation (ChIP) assays (FIGS. 3B and 3C). To evaluate the effect of androgen receptor on the expression of tyrosine hydroxylase, the synthesis of endogenous androgen receptor was reduced using androgen receptor specific siRNA constructs and the protein levels of tyrosine hydroxylase was examined. Inhibition of the androgen receptor expression resulted in a gradual decrease in the level of endogenous tyrosine hydroxylase (FIG. 4) These results are consistent with androgen receptor acting as a transcriptional activator and with the observation that the tyrosine hydroxylase promoter is positively regulated by DJ-1.

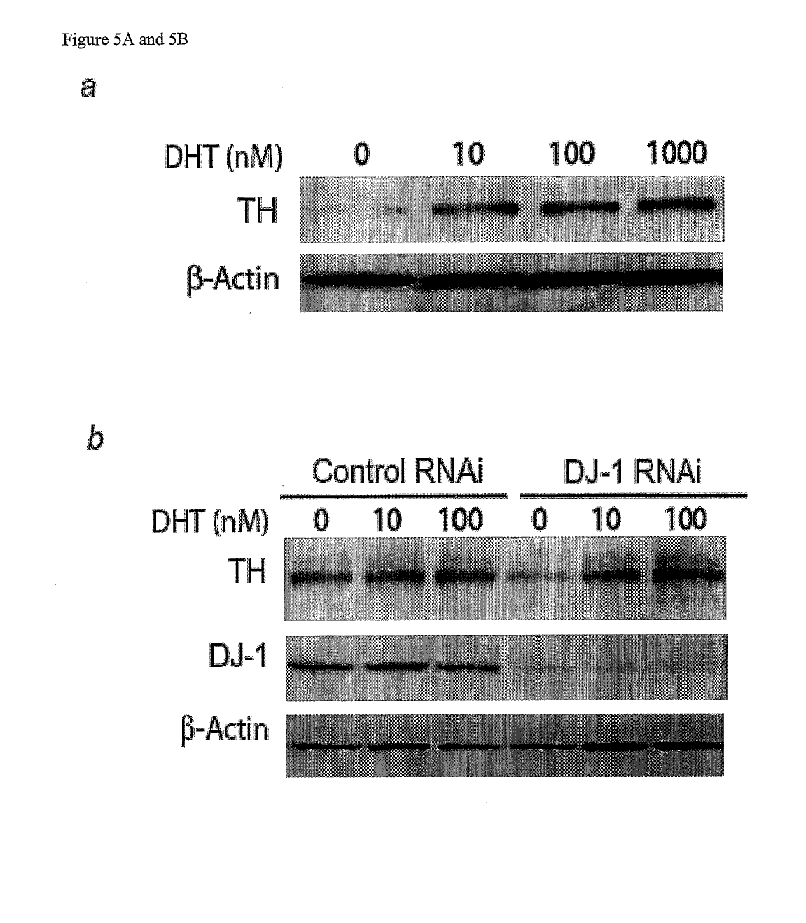

Since the transcriptional activities of androgen receptor are triggered by its ligands, such as testosterone or its derivatives⁵³, untransfected CHP-212 cells were treated with dihydrotestosterone for forty-eight hours and the expression of tyrosine hydroxylase was measured. Dihydroxytestosterone treatment led to a dose-dependent increase in tyrosine hydroxylase protein levels (FIG. 5A). To evaluate whether dihydroxytestosterone could reverse the loss of tyrosine hydroxylase caused by DJ-1 inactivation, CHP-212 cells were pre-transfected with control or DJ-1-specific siRNA then treated with increasing amount of dihydroxytestosterone for forty-eight hours. Protein levels of tyrosine hydroxylase and DJ-1 in these cells was subsequently assayed. While DJ-1-specific siRNA depleted cellular DJ-1, dihydroxytestosterone treatment fully rescued tyrosine hydroxylase expression (FIG. 5B). Taken together, these results indicated that androgen receptor transcriptionally activated human tyrosine hydroxylase. These results also indicated that dihydroxytestosterone and androgen receptor signaling likely counteracted DJ-1 inactivation-induced transcriptional inhibition of the tyrosine hydroxylase promoter.

### Example 4

### Androgen Treatment Activated the Pro-Survival AKT Pathway and Alleviated Oxidative Stress-Induced Cell Death in Human Dopaminergic Neuroblastoma Cell Lines

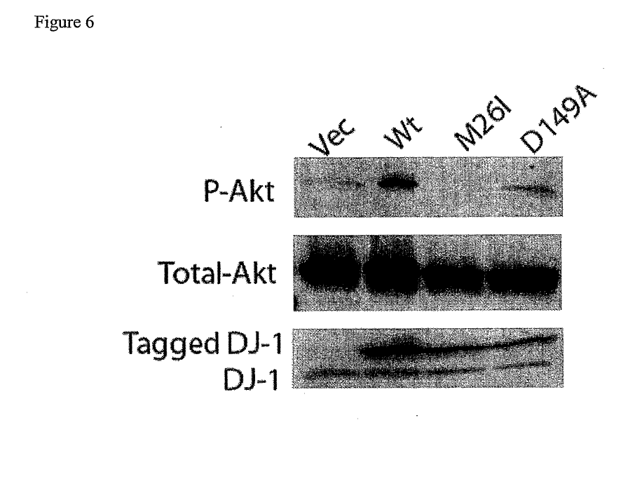

Functional studies clearly indicate that DJ-1 is a neuroprotective protein⁵⁴⁻⁵⁹. The neuroprotective activity of DJ-1 has been attributed, in part, to its ability to regulate oxidative stress response^{58,59}, protein folding⁵⁶, and transcription⁵⁷. In addition, DJ-1 has been functionally linked to the AKT signaling pathway in vivo^{60,61}. The phosphorylation of AKT promotes its catalytic activity and triggers a signal transduction cascade to stimulate cell growth and survival⁶². To assay AKT pathway activation of DJ-1 in human dopaminergic cells, SH-SY5Y cells that stably express the wild-type or pathogenic DJ-1 mutants were grown in culture, and phosphorylated AKT levels were assayed. The overexpression of wild-type DJ-1 in this dopaminergic neuroblastoma cell line resulted in increased phosphorylation of AKT (FIG. 6). Increased AKT phosphorylation was not observed in SH-SY5Y cells expressing the mutant form of DJ-1 that is clinically associated with early-onset Parkinsonism (M26I and D149A) (FIG. 6). The inability of the pathogenic DJ-1 mutants to activate the AKT pathway suggested that abnormal AKT signaling is likely one underlying mechanism that contributes to Parkinson disease pathogenesis.

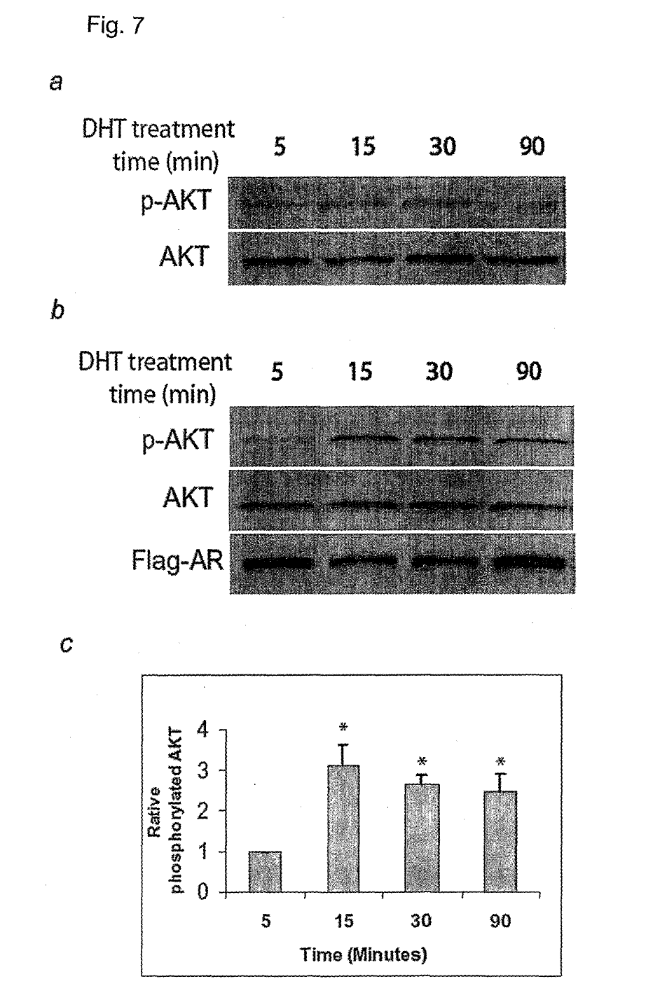

DJ-1 likely serves as a transcriptional co-activator of androgen receptor^{50,51}. To determine whether androgen receptor and androgen activate the AKT pathway in human dopaminergic cell lines, native SH-SY5Y cells were treated with 10 nM of dihydroxytestosterone, and levels of phosphorylated AKT was assayed by Western blot. Dihydroxytestosterone treatment resulted in the rapid phosphorylation of AKT without increasing the expression of total AKT (FIG. 7A). The amount of phosphorylated AKT reached peak level ninety minutes after dihydroxytestosterone treatment. The role of androgen receptor signaling in the activation of AKT was also tested in SH-SY5Y cells that were transiently transfected with human androgen receptor then treated with dihydroxytestosterone. Androgen receptor expression accelerated AKT phosphorylation and shifted the peak level of phosphorylated AKT to 15 minutes after dihydroxytestosterone treatment (FIGS. 7B and 7C). These results indicate that androgen receptor and DJ-1 share a similar signaling pathway that promotes neuronal survival.

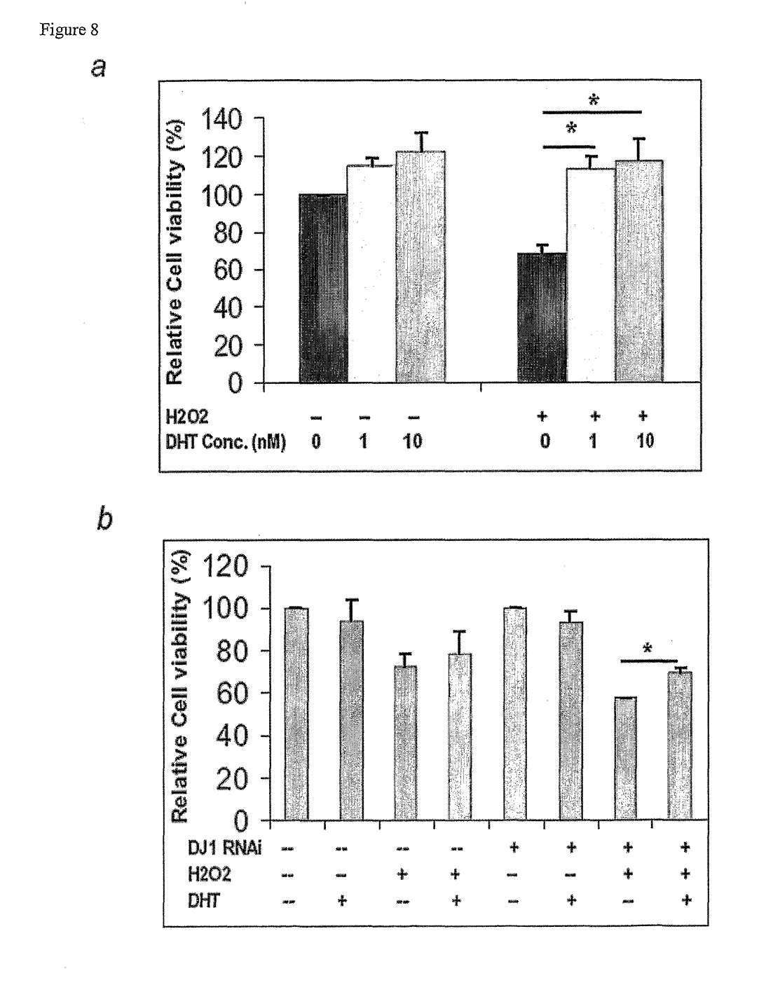

Increasing evidence suggests that DJ-1 functions in neuroprotection and cellular defense against oxidative stress⁶⁴. To evaluate whether dihydroxytestosterone similarly protects against oxidative stress-induced cell death in human dopaminergic neuroblastoma cells, SH-SY5Y cells were treated with increasing amount of dihydroxytestosterone in the presence or absence of 500 μM of hydrogen peroxide (H₂O₂). Although dihydroxytestosterone had a subtle effect on cell viability in the absence of H₂O₂, dihydroxytestosterone significantly blocked cell death induced by oxidative stress (FIG. 8A). Loss of DJ-1 rendered cells more susceptible to oxidative stress. To test whether dihydroxytestosterone can reverse this increased susceptibility, endogenous DJ-1 expression in CHP-212 cells was inhibited using DJ-1-specific siRNA. This inhibited DJ-1 expression by 70% as confirmed by western blot. The siRNA treated cells were then further treated with 10 nM of dihydroxytestosterone in the presence or absence of 300 μM of H₂O₂. Consistent with results in a DJ-1-deficient mouse⁶⁵, DJ-1 inactivation enhanced cellular sensitivity to oxidative stress (FIG. 8B, graph bar 7 (+DJ1 RNAi, +H₂0₂; −DHT) vs. graph bar 3 (−DJ1 RNAi, +H₂O₂, −DHT)). Dihydroxytestosterone treatment significantly reduced oxidative stress-induced cell death (FIG. 8B, graph bar 8 (+DJ1 RNAi, +H₂0₂; +DHT) vs. graph bar 7 (+DJ1 RNAi, +H₂0₂; −DHT) in DJ-1-specific siRNA treated cells. These observations indicate that androgen is neuroprotective in human dopaminergic cells, and prevents neuronal cell death caused by DJ-1 inactivation.

The results reported herein indicate a role for DJ-1 in the cellular defense against oxidative stress, which is a major contributor to neurodegenerative diseases⁶⁴. Loss of DJ-1 results in the degradation of a master regulator of the antioxidant transcriptional response, Nrf2⁶⁶. Even though mutations in the DJ-1 gene are rare, several recent studies suggest that DJ-1 may be functionally inactivated by age or disease-related oxidative damage⁶⁷⁻⁶⁹. Therefore, the normal function of DJ-1 is likely to be an essential component in the battle against accumulated oxidative insults and the neuronal survival during aging or the progression of the neurodegenerative diseases. Besides the regulation of the stress response and cell survival pathway, DJ-1 transcriptionally up-regulates the expression of the human tyrosine hydroxylase, the rate-limiting enzyme for dopamine synthesis. Thus, DJ-1 function is important for neuronal survival and for dopaminergic function, both of which are reduced Parkinson's disease.

The results describe here suggest that androgen receptor signaling reverses deficits in tyrosine hydroxylase synthesis and in the cell survival pathway caused by DJ-1 inactivation. The androgen receptor is required for the expression of tyrosine hydroxylase, and androgen reverses the loss of tyrosine hydroxylase caused by DJ-1 inactivation. In addition, both DJ-1 and dihydroxytestosterone stimulate the activation of the neuroprotective AKT pathway, and dihydroxytestosterone reduces dopaminergic cell death associated with oxidative stress. These data support a functional links between the androgen receptor and DJ-1^{49,50}. The results reported herein provide new molecular evidence supporting the regulation of dopaminergic function and cell survival by the male sex hormone, and describe common pathways governed by androgen receptor and DJ-1. This indicates that androgen replacement therapy is likely to be beneficial for the treatment or prevention of dopaminergic cell loss, particularly in male patients with Parkinson's disease.

The experiments described above were carried out as follows.

### Cell Culture, Plasmids and Chemicals.

Human CHP-212 cells were purchased from ATCC and maintained in cell culture media, EMEM/F-12 (50%/50%) containing 10% FBS and antibiotics. Native SH-SY5Y cells were maintained in DMEM supplemented with 10% FBS and antibiotics. For immunofluorescence, cells were grown on coverslips. Wild-type and mutant DJ-1 constructs and SH-SY5Y cells stably expressing these constructs were described previously¹⁸. Rat tyrosine hydroxylase-luc reporter plasmid (Kim et al Biochem Biophys Res Commun. 2003 Dec. 26; 312(4):950-7) and the pTK-Renilla luciferase plasmid for transfection control were obtained from Promega (Madison, Wis.). Human androgen receptor expression construct PCMV-Flag-AR was kindly provided by Dr. E. Wilson⁷⁵. H₂O₂ and DHT were purchased from Sigma (St. Louis, Mo.).

Transfection of siRNA and Plasmids.

CHP-212 cells or SH-SY5Y cells were plated in six-well culture dishes and transfected with 100 nM of siRNA against human DJ-1 constructs (SMARTpool reagent, Dharmacon, Lafayette, Colo.) or non-specific control siRNA constructs (siControl non-targeting pool, Dharmacon). The siRNA was transfected in to cells using the cationic lipid Transfectin reagent (Bio-Rad, Hercules, Calif.) following the manufacturer's suggested protocol. Cells were harvested forty-eight hours post-transfection for RNA extraction or at Day 1,2, or 4 for Western blot or re-plated in 96 well plate for MTS assay at 48 hrs post-transfection. To analyze the effects of dihydroxytestosterone on tyrosine hydroxylase expression after DJ-1 inactivation, various amounts of DHT were added to fresh medium at forty-eight hours post-transfection. Cells were cultured for an additional two days with medium containing dihydroxytestosterone before being harvested for Western blot.

To analyze the effects of androgen on the tyrosine hydroxylase expression after DJ-1 inactivation, the indicated amount of dihydroxytestosterone was added to fresh medium at forty-eight hours post-transfection and cultured for an additional two days with one change of fresh medium containing DHT. Lipofectanine 2000 (Invitrogen, Carlsbad, Calif.) was used to transfect various plasmids.

### Western Blotting, Immunoprecipitation and Antibodies.

The procedures for western blotting and immunoprecipitation were described previously⁴⁸. For DJ-1 immunoprecipitation, cells were lysed in denaturing RIPA-DOC buffer (50 mm Tris-HCl buffer (pH 8.0), containing 150 mM NaCl, 1% Triton X-100, 0.1% SDS, 1% sodium deoxycholate, 10 mM EDTA and a protease inhibitor cocktail (1× protease cocktail, Roche (Indianapolis, Ind.). For endogenous DJ-1 and PSF co-immunoprecipitation, cells were lysed in non-denaturing lysis buffer containing 1% Triton-X100. For endogenous DJ-1 co-immunoprecipitation, cells were lysed in non-denaturing lysis buffer containing 1% Triton-X100. Antibody used for immunoprecipitation: rabbit polyclonal anti-DJ-15. Antibodies for western blotting include: a mouse monoclonal anti-tyrosine hydroxylase (Sigma); monoclonal (Stressgen, San Diego, Calif.) and polyclonal anti-DJ-1; a goat anti-β-actin (Santa Cruz Biotechnology, Santa Cruz, Calif.); a rabbit polyclonal anti-androgen receptor (Upstate, Charlottesville, Va.); a rabbit polyclonal anti-AKT and a mouse monoclonal anti-Phospho-AKT (Cell signaling, Beverly, Mass.). Antibodies used for immunoprecipitation included a mouse monoclonal anti-PSF (Sigma) and a rabbit polyclonal anti-DJ-1¹⁸. Antibodies for western blotting included a mouse monoclonal anti-tyrosine hydroxylase (Sigma); monoclonal (Stressgen, San Diego, Calif.) and polyclonal anti-DJ-1 a rabbit polyclonal anti-acetylated histones (Histone sampler kit, Cell signaling, Beverly, Mass.); and a rabbit polyclonal anti-androgen receptor antibody.

### RNA Extraction and Real-Time Quantitative PCR (Q-PCR).

RNA was extracted using a mono-phasic solution of phenol and guanidine isothiocyanate that is commercially available as Trizol reagent (Invitrogen) and purified with a commercially available silica-gel-based membrane, the RNeasy Kit or RNeasy Micro Kit (Qiagen, Germany), and quantified with a spectrophotometer. The quality of RNA was confirmed by agarose gel electrophoresis. The reference RNA used for calibration curve was made by pooling equal amount of RNA from all samples. Q-PCRs were performed using a LightCycler (Roche, Indianapolis, Ind.) and One-Step QuantiTect™ SYBR Green RT-PCR kit (Qiagen) that provides for kinetic quantification of PCR products. Kinetic quantification of real-time PCR allows the course of a polymerase chair reaction to be visualized as a curve that contains an initial lag phase, an exponential (log-linear) phase, and a final plateau phase. Experimental conditions and primer design parameters were set in accordance with the manufacturer's instructions. Primers for Q-PCR were designed to have an amplicon size of 100-200 bps. Agarose gel electrophoresis was used to confirm the specificity of PCR reactions. Results were normalized to an internal control PCR amplified with GAPDH or β-Actin primers included in the same run of Q-PCR. Primers for the human tyrosine hydroxylase: Forward: 5′-cctcgcccatgcactc-3′; Reverse: 5′-cctcgcccatgcactc-3′.

### Chromatin Immunoprecipitation (ChIP) Assays.

Chromatin immunoprecipitation (ChIP) assays were performed using a commercially available kit, the EZ ChIP Kit (Upstate, Charlottesville, Va.), that includes lysis buffer to lyse formaldehyde-treated cells prior to sonication, a protein A agarose slurry that precipitates antibody-protein-DNA complexes, several wash buffers that are necessary for reducing non-specific background interactions, and a 5M NaCl solution that reverses the formaldehyde cross-links in accordance with the manufacturer's instructions with the following modifications. After protein-DNA cross-linking and harvesting, the cell pellets were resuspended in lysis buffer and sonicated on ice using a Branson Digital Sonifier (Branson Ultrasonics Corporation, CT) with 16 sets of 4-second pulses at 17% of maximum power. The genomic DNA was sheared to 300-1200 bp in length. Aliquots of chromatin solution (each equivalent to 1×10⁶ cells) were precleared with Protein G agarose and incubated with species-matched IgG or specific antibodies overnight at 4° C. with rotation. The antibodies used in the ChIP assays for DJ-1, PSF and acetylated histones were described above. The final immunoprecipitated DNA fragments were used as templates for PCR with a commercially available recombinant Taq DNA polymerase complexed with a proprietary antibody that blocks polymerase activity at ambient temperatures, hot start Platinum Taq, (Invitrogen, San Diego, Calif.) using the following conditions: 3 minutes at 94° C.; 32 cycles of 30 seconds denaturation at 95° C., 30 seconds annealing at 57° C. and 30 seconds elongation at 72° C.; with one final incubation for 2 minutes at 72° C. For semi-quantitative PCR, 27, 29, 31 and 33 cycles were used. The Primer 3 software was used to design the PCR primers for amplifying the human tyrosine hydroxylase promoter. The primers for ChIP using anti-DJ-1, and acetyl-histones: Forward: 5′-gagccttcctggtgtttgtg-3′, and reverse: 5′-ctctccgattccagatggtg-3′. The primers for ChIP using anti-AR: Forward: 5′-gggtcttccctttgctttga-3′, and reverse: 5′-cctgggacctttcctaaaactg-3′. The PCR products were analyzed by electrophoresis on commercially available 2% TAE agarose gels.

### Statistical Analysis.

Statistical analyses were performed using InStat 3.0 (GrapbPad, San Diego, Calif.). P values, sample numbers and statistical tests used were indicated in the figure legends.

A review of the following specific references will help advance appreciation of the present invention.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

### REFERENCES

1. Morris, H. R., (2005) Ann Med 37:86-96
2. Cookson, M. R., (2003) Neuron 37:7-10
3. Dawson, et al., (2003) Science 302:819-22
4. Bonifati, et al. (2003) Science 299:256-9
5. Bonifati, et al., (2004) J Mol Med
6. Nagakubo, et al., (1997) Biochem Biophys Res Commun 231:509-13
7. Wagenfeld, et al., (2000) 21:954-63
8. Okada, et al., (2002) Biol Pharm Bull 25:853-6
9. Takahashi, et al., (2001) J Biol Chem 276:37556-63
10. Niki, et al., (2003) Mol Cancer Res 1:247-61
11. Mitsumoto, et al. Free Radic Res 35:301-10
12. Taira, et al. (2004) EMBO Rep 5:213-218
13. Wilson, et al, (2003) Proc Natl Acad Sci. USA 100:9256-61
14. Tao, et al., (2003) J Biol Chem 278:31372-9
15. Honbou, et al. (2003) J Biol Chem 278:31380-4
16. Huai, et al., (2003) FEBS Lett 549:171-5
17. Miller, et al., (2003) J Biol Chem 278:36588-95
18. Xu, et al., (2005) Hum Mol Genet. 14:1231-1241
19. Takahashi-Niki, (2004) Biochem Biophys Res Commun 320:389-97
20. Macedo, et al., (2003) Hum Mol Genet. 12:2807-16
21. Moore, et al., (2003) J Neurochem 87:1558-67
22. Moore, et al., (2005) Hum Mol Genet. 14:71-84
23. Yokota, et al., Biochem Biophys Res Commun 312:1342-8
24. Canet-Aviles, et al., (2004) Proc Natl Acad Sci USA 101:9103-8
25. Martinat, et al., (2004) PLoS Biol 2, e327
26. Kim, et al., (2005) Cancer Cell 7:263-73
27. Junn, et al., (2005) Proc Natl Acad Sci USA 102:9691-6
28. Olzmann, et al., (2004) J Biol Chem 279:8506-15
29. Shendelman, et al., (2004) PLoS Biol 2, e362
30. Goldberg, et al., (2005) Neuron 45:489-496
31. Kim, et al., (2005) Proc Natl Acad Sci USA 102:5215-20
32. Chen, et al., (2005) J Biol Chem 280:21418-26
33. Meulener, et al., (2005) Curr Biol 15:1572-7
34. Menzies, et al., (2005) Curr Biol 15:1578-82
35. Yang, et al., (2004) Proc Natl Acad Sci USA
36. Johnson, E. S., (2004) Annu Rev Biochem 73:355-82
37. Melchior, et al., (2003) Trends Biochem Sci 28:612-8
38. Gill, G., (2004) Genes Dev 18:2046-59
39. Girdwood, et al., (2004) Semin Cell Dev Biol 15:201-10
40. Shinbo, et al., (2005) Cell Death Differ
41. Ross, C. A., (2002) Neuron 35:819-22
42. Okazawa, H., (2003) Cell Mol Life Sci 60:1427-39
43. Steffan, et al., (2004) Science 304:100-4
44. Rosas-Acosta, et al., (2005) Mol Cell Proteomics 4:56-72
45. Ishitani, et al., (2003) Biochem Biophys Res Commun 306:660-5
46. Black, et al., (2004) Trends Endocrinol Metab 15:411-7
47. Maharjan, et al., (2005) J Neurochem 93:1502-14
48. Xu, et al., (2002) Nat Med 8:600-6
49. Zhong, et al., (2006) J Biol Chem 281, 20940-8.
50. Niki, et al., (2003) Mol Cancer Res 1, 247-61.
51. Takahashi, et al., (2001) J Biol Chem 276, 37556-63.
52. Ishitani, et al., (2003) Biochem Biophys Res Coinmun 306, 660-5.
53. Black, et al., (2004) Trends Endocrinol Metab 15, 411-7.
54. Canet-Aviles, et al., (2004) Proc Natl Acad Sci USA 101, 9103-8.
55. Junn, et al., (2005) Proc Natl Acad Sci USA 102, 9691-6.
56. Shendelman, et al., (2004) PLoS Biol 2, e362.
57. Xu, et al., (2005) Hum Mol Genet. 14, 1231-1241.
58. Taira, et al., (2004) EMBO Rep 5, 213-218.
59. Menzies, et al., (2005) Curr Biol 15, 1578-82.
60. Kim, et al., (2005) Cancer Cell 7, 263-73.
61. Yang, et al., (2005) Proc Natl Acad Sci USA.
62. Cantley, et al., Science (2002) 296, 1655-7.
63. Abou-Sleiman, et al., (2003) Ann Neurol 54, 283-6.
64. Cookson, et al., (2005) Annu Rev Biochem 74, 29-52.
65. Martinat, et al., (2004) PLoS Biol 2, e327.
66. Clements, et al., (2006) Proc Natl Acad Sci USA.
67. Zhou, et al., (2006) J Mol Biol 356, 1036-48.
68. Choi, et al., (2006) J Biol Chem 281, 10816-24.
69. Meulener, et al., (2006) Proc Natl Acad Sci USA 103, 12517-22.
70. Van Den Eeden, et al., (2003) Am J Epidemiol 157, 1015-22.
71. Ready, et al., (2004) J Neurol Neurosurg Psychiatry 75, 1323-6.
72. Okun, et al., (2004) Neurology 62, 411-3.
73. Alam, et al., (2004) Physiol Behav 83, 395-400.
74. Okun, et al., (2002) Arch Neurol 59, 1750-3.
75. He, et al., (2006) J Biol Chem 281, 6648-63.

1. A method for reducing neuronal cell death associated with a neurodegenerative disease, the method comprising contacting a cell at risk of cell death with an effective amount of an androgen or androgen analog thereby reducing neuronal cell death.

2. The method of claim 1, wherein the method increases tyrosine hydroxylase expression in the cell.

3. (canceled)

4. A method for reducing oxidative stress in a cell in need thereof, the method comprising contacting the cell with an androgen or androgen analog, thereby reducing oxidative stress.

5-6. (canceled)

7. A method for increasing tyrosine hydroxylase expression in a neuronal cell in need thereof, the method comprising contacting the cell with an effective amount of an agent that increases DJ-1 expression or activity.

8. The method of claim 7, wherein the agent is valproic acid, sodium butyrate, trichostatin A, or SAHA

9. The method of claim 1, wherein the method increases tyrosine hydroxylase transcription or translation.

10-17. (canceled)

18. A method for preventing or treating a neurodegenerative disease in a subject in need thereof, the method comprising administering to the subject an effective amount of an androgen that increases the expression or activity of tyrosine hydroxylase thereby preventing or treating the neurodegenerative disease.

19. (canceled)

20. The method of claim 18, wherein the neurodegenerative disease is Parkinson's Disease.

21. The method of claim 18, wherein administering to the subject an effective amount of an androgen or androgen analog increases dopamine synthesis.

22. The method of claim 18, wherein the neurodegenerative disease is selected from the group consisting of Parkinson's disease, Huntington's Disease, Kennedy's Disease, and spinocerebellar ataxia, and the method reduces cell death in the subject.

23-33. (canceled)

34. A packaged pharmaceutical comprising:
(a) an androgen or androgen analog; and
(b) instructions for using said androgen to treat a neurodegenerative disease.

35. The packaged pharmaceutical of claim 34, further comprising a therapeutic selected from the group consisting of deprenyl, amantadine, levodopa, carbidopa, entacapone, pramipexole, rasagiline, antihistamines, antidepressants, dopamine agonists, monoamine oxidase inhibitors (MAOIs), haloperidol, phenothiazine, reserpine, tetrabenazine, and co-enzyme Q10.

36. A method for identifying a compound useful for the treatment of a neurodegenerative disease, the method comprising:
(a) contacting a neuronal cell with a compound and an androgen receptor agonist; and
(b) identifying an increase in the expression of a gene of interest in the cell relative to a control cell not contacted with the candidate compound, wherein a compound that increases the expression of a gene of interest is a compound useful for the treatment of a neurodegenerative disease.

37-47. (canceled)

48. A method for identifying a gene required for neuronal survival or maintenance that is transcriptionally activated by androgen receptor binding, the method comprising:
(a) contacting a cell expressing the gene with an androgen receptor agonist; and
(b) identifying binding of the androgen receptor to a regulatory sequence present in the gene or identifying an increase in expression of the gene.

49. A method for identifying a gene required for neuronal survival or maintenance that is transcriptionally activated by androgen receptor binding, the method comprising:
(a) contacting a cell expressing the gene with an androgen receptor agonist; and
(b) identifying an increase in expression of the gene in the cell relative to a control cell not contacted with the androgen receptor agonist.

50. The method of claim 48, wherein the androgen is testosterone, dihydrotestosterone (DHT), or an analog or fragment thereof.

51. The method of claim 48, wherein the cell is a mammalian cell.

52. The method of claim 51, wherein the cell is a human cell.

53. The method of claim 51, wherein the increase in expression is detected by means of a detectable reporter.

54. The method of claim 51, wherein the detectable reporter is operably linked to the gene.
